(19) United States
(12) Patent Application Publication
(10) Pub. No.: US 2004/0215122 A1
(43) Pub. Date: Oct. 28, 2004

(54) Leg support device for surgical patients

(76) Inventor: Stephen Hansen, Omaha, NE (US)

Correspondence Address: Law Offices of Adam H. Jacobs, Suite 726, 1904 Farnam Street, Omaha, NE, 68102, US

(21) Appl. No.: 10/831,257

(22) Filed: Apr. 23, 2004

Related U.S. Application Data
(60) Provisional application No. 60/464,907, filed on Apr. 23, 2003.

(51) Int. Cl. A61F 5/00

(52) U.S. Cl. 602/23

(57) ABSTRACT

An extremity support device including a box constructed of a rigid, non-permeable material, the box being generally rectangular in shape and including at least two sides having concave surfaces for releasably retaining a patient's extremity thereon, the rectangular box having different height, width, and length dimensions to permit support of the patient's extremity at different heights for different caregiver purposes.

### CROSS-REFERENCE TO RELATED PROVISIONAL PATENT

This application claims priority based on a provisional patent, specifically on the Provisional Patent Application Serial No. 60/464,907 filed Apr. 23, 2003.

### BACKGROUND OF THE INVENTION

1. Technical Field

The present invention is directed to surgery assist devices and, more particularly, to a leg support device for surgical patients which has a generally rectangular box-like shape and includes at least two sides having concave surfaces for releasably retaining a patient's leg thereon, the rectangular box having different height, width, and length dimensions to permit support of the patient's leg at different heights.

2. Description of the Prior Art

A large percentage of surgeries performed are to the human extremities, in other words, the arms, legs, hands and feet of the person on whom the operation is being performed. Very often in performing these operations, it is necessary to elevate the extremity in order to more easily access the location on which the surgery is to be performed. It is important, however, that whatever the means used to elevate the extremity, it must be at least as sterile as the rest materials in the operating suite in order to ensure that the patient will not be infected by microbial or viral infectants. In the past, this elevation of the extremity was performed by stacking a number of sterile towels on top of one another until the desired height for supporting the extremity was reached. However, with upwards of ten towels being stacked on one another, it is seen that the resulting towel stack is quite unstable and can easily collapse, thus subjecting the extremity to a traumatic fall which can cause further injury to the extremity. There is therefore a need for a safe and sturdy support for extremities which can be used in an operating suite environment.

It has been proposed to use other types of support structures, such as plastic or fabric supports which are internally braced. These devices, however, include one major inherent deficiency, and that is that they are easily contaminated by bodily fluids released from the extremity on which the operation is being performed. Furthermore, there is no easy way by which the support devices may be cleaned and thus the same problem encountered with the towels is encountered with these devices in that they may not be reused without a thorough cleaning being applied thereto, and such thorough cleaning may not be able to be applied. There is therefore a need for an extremity support device which may be quickly and easily cleaned following its use with a patient.

The majority of instruments used in the operating suite are of a high quality stainless steel or aluminum, and these are designed in such a way that they may be inserted into a device known as an “autoclave” for cleaning thereof. The autoclave is designed to heat the metal instruments to extremely high temperatures and keep them at those temperatures for an extended period of time in order to completely sterilize the metal instruments. The autoclave is standard equipment in virtually every operating room and is quickly and easily used by even the least experienced of doctors and nurses. There is therefore a need for an extremity support device which can be safely placed in the autoclave for sterilizing to permit the quick and simple reuse of the extremity support device in the operating room.

Therefore, an object of the present invention is to provide an improved extremity support device.

Another object of the present invention is to provide an improved extremity support device which includes a generally rectangular box having at least two sides having concave surfaces for releasably retaining a patient's extremity thereon, the box being constructed of a rigid, non-permeable material such as stainless steel or aluminum.

Another object of the present invention is to provide an improved extremity support device which includes different height, width, and depth measurements such that rotation of the box to rest the box on a different side results in the topmost wall surface being supported at a different height to permit the easy adjustability of the height of the support surface for the extremity.

Another object of the present invention is to provide an improved extremity support device which is constructed of metal which can be inserted into an autoclave device for efficient sterilization of the extremity support device.

Finally, an object of the present invention is to provide an improved extremity support device which is relatively simple in construction and is safe and efficient in use.

### SUMMARY OF THE INVENTION

The present invention provides an extremity support device including a box constructed of a rigid, non-permeable material, the box having at least six sides, and a first set of two sides of the six sides being positioned generally opposite and generally parallel with one another and having generally similar lengths. A second set of two sides of the six sides is also positioned generally opposite and generally parallel with one another and has generally similar lengths. A first side of the first set includes at least a portion thereof which is generally flat such that the box resting on the first side of the first set on a support surface is generally stable, and a second side of the first set includes a generally concave wall surface extending substantially the length of the second side and having a generally cross-sectionally concave shape generally adapted to receive and releasably retain an extremity of an animal therein. A first side of the second set includes at least a portion thereof which is generally flat such that the box resting on the first side of the second set on a support surface is generally stable, and a second side of the second set includes a generally concave wall surface extending substantially the length of the second side and having a generally cross-sectionally concave shape generally adapted to receive and releasably retain an extremity of an animal therein. Finally, the length of the sides of the first set is different from the length of the sides of the second set such that the second side of the first set is supported at one height above the first side of the first set when the box is resting on the first side of the first set and the second side of the second set is supported at another height above the first side of the second set when the box is resting on the first side of the second set.

The present invention is specifically designed to address and correct the deficiencies of the prior art and provide an easily used and safe device for elevating an extremity (arm or leg) of an animal such as a human being. For example, due to the concave design of at least two of the walls of the box, the extremity is “nestled” into the concavity thus safely, securely and comfortably supporting the extremity allowing improved access to the extremity for the purpose intended by the caregiver. Furthermore, because each of the sets of sides of the box is of different lengths, rotation of the box will permit the extremity to be supported by a different side which is positioned at a different height than the other sides, which permits the present invention to be used in many more situations than that permitted with those inventions found in the prior art. Finally, because the present invention is constructed of a rigid, nonpermeable material, it may be quickly and easily cleaned in an autoclave or the like for use with another patient, unlike those devices found in the prior art. The present invention thus provides a substantial improvement over those devices found in the prior art.

### BRIEF DESCRIPTION OF THE DRAWINGS

FIG. 1 is a perspective view of the leg support device of the present invention being used with a patient;

FIG. 2 is a perspective view of the leg support device of the present invention showing the concave surface of the side walls of the rectangular box;

FIG. 3 is a perspective view of the leg support device of the present invention taken at a different angle;

FIG. 4 is a perspective view of an alternative embodiment of the leg support device of the present invention taken at a different angle; and

FIG. 5 is a perspective view of a second alternative embodiment of the leg support device of the present invention taken at a different angle.

### DESCRIPTION OF THE PREFERRED EMBODIMENT

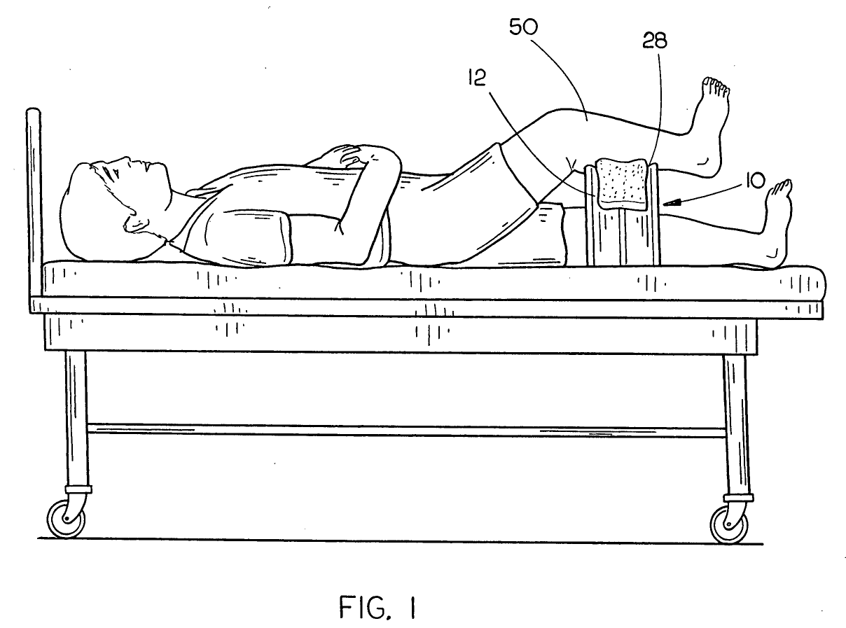

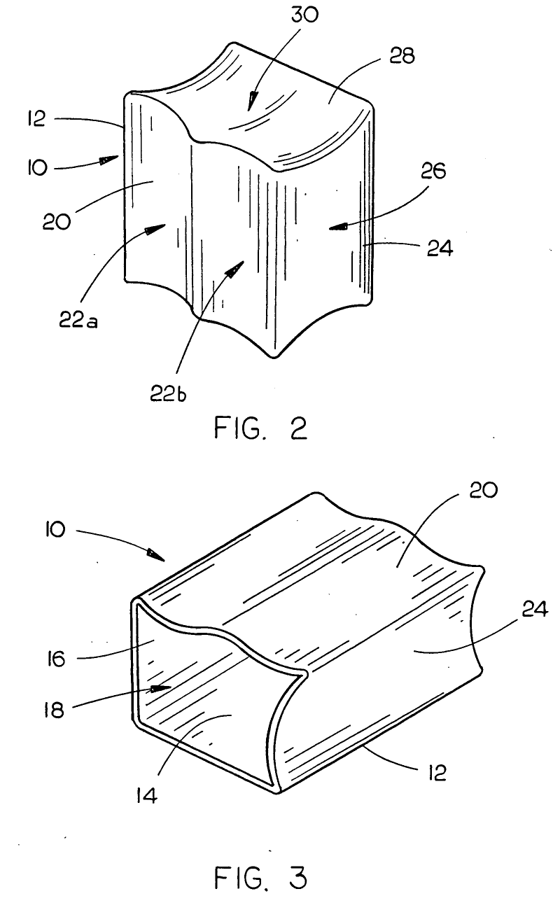

The leg support device 10 of the present invention shown best in FIGS. 1-3 as including a generally rectangular box structure 12 which, in the preferred embodiment, would be constructed of a high-grade metal such as stainless steel or aluminum. Although the rectangular box 12 may be constructed of other materials, it is preferred that the construction material of the rectangular box 12 be a metal as described above in order to permit easy sterilization of the rectangular box 12 and hence the leg support device 10 by insertion of the rectangular box 12 into an autoclave or the like which are commonly used in sterilizing surgical equipment.

In one preferred embodiment, the rectangular box 12 would have dimensions of approximately six to ten inches in height, four to six inches in width, and four to eight inches in length, with each of the dimensions of height, width, and length being different in order to enhance the usability of the present invention. Of course, there may be instances where it is important to have the dimensions of the rectangular box 12 be identical or outside of the above-described dimensions, depending upon the use of the present invention, but it has been found that as a general rule, the above dimensions provide sufficient variation in the size of the rectangular box 12 to permit its use and enhance its functionality.

As shown best in FIG. 1, the rectangular box 12 would include two generally flat planar base walls 14 and 16 and a base opening 18 which generally functions as a third base wall when the rectangular box 12 is being used. Because each of the large base wall 14, small base wall 16, and base opening 18 are generally flat, they each provide a stable planar surface on which the rectangular box 12 can rest, thus enhancing the stability of the rectangular box 12 and permitting use of the leg support device 10 for supporting of a patient's arm or leg thereon. It should be noted that the dimensions of the large base wall 14, small base wall 16, and base opening 18 are not critical to the present invention so long as they provide a generally stable surface on which the rectangular box 12 can rest.

First concave support wall 20 is directly opposite large base wall 14 and would include one or more extended concave sections 22a and 22b formed by the first concave support wall 20. Due to the relatively large size of first concave support wall 20, it has been found that the inclusion of two concave sections 22a and 22b are preferable to having a single concavity formed in the first concave support wall 20, as the majority of patients' legs are too narrow to fit comfortably within such a large concavity. Of course, for larger patients, the use of such a large concavity may be desirable, and such a modification is contemplated by the present invention. It is further preferred that the concave sections 22a and 22b be formed as relatively gentle wavelike structures in order to enhance the comfort level of the patient whose leg is being supported by the leg support device 10 of the present invention, yet still retain the patient's leg 50 within the concave section 22a and 22b in which it is resting.

Second concave support wall 24 is directly opposite small base wall 16 and includes a single concavity 26 in which the patient's leg 50 may rest and third concave support wall 28 is directly opposite base opening 18 and would also include a single concavity 30 in which the patient's leg 50 may rest. The depths of each of the concavities 26 and 30 are critical to the present invention only in that the depths are sufficient to retain the patient's leg 50 within the concavity 26 and 30 when the patient's leg 50 is resting on the leg support device 10. To this end, it has been found that the depth of each concavity 26 and 30 should be between one-half and one inch, although, as was stated previously, the exact dimensions are not critical to the present invention so long as the functional characteristic of retaining the patient's leg within the concavity 26 and 30 is maintained.

One of the unique aspects of the present invention is that the patient's leg 50 may be supported at three different heights by the leg support device 10 of the present invention by merely rotating the rectangular box 12 to present a selected one of the first concave support wall 20, second concave support wall 24 and third concave support wall 28 to the patient's leg 50. As shown in FIGS. 1-3, the use of the first concave support wall 20 for supporting the patient's leg 50 would raise the patient's leg 50 approximately four inches above the bed, gurney, or floor on which the patient is resting. This height is ideal for many surgical applications, and the dual concave sections 22a and 22b of first concave support wall 20 permit the patient's leg 50 to be moved closer to or farther away from the surgeon depending on the desired location of the patient's leg 50 to facilitate the surgical procedure. Should the surgeon require the patient's leg 50 to be elevated to a higher position, the patient's leg 50 may be lifted and the rectangular box 12 rotated to present the second concave support wall 24 to the patient's leg 50. The rectangular box would thus be resting on the small base wall 16 and the approximate height of the second concave support wall 24 above the bed or gurney would be six inches. At this height, it is easier for a surgeon or x-ray technician to access the leg for viewing of the leg features and x-raying of those features. Finally, should the patient's leg 50 require additional elevation, the patient's leg 50 would be lifted and rectangular box 12 would be rotated to rest on the base opening 18, thus presenting the third concave wall 28 to the patient's leg 50 so that the patient's leg 50 may rest thereon. The patient's leg 50 would be elevated approximately eight inches or so above the bed or gurney on which it is resting, thus permitting even easier access to the patient's leg 50 by the surgeon and/or x-ray technician. The adjustment in height of the patient's leg 50 is thus shown to be extremely simple in connection with the use of the present invention, a feature not found in those devices of the prior art.

In fact, the previous method of elevating a patient's leg during surgery was to place a number of sterile towels underneath the leg to prop it up to the desired height. This often required in excess of five to ten folded towels to accomplish the same level of elevation as provided by the leg support device 10 of the present invention. With use of the leg support device 10 of the present invention, it is preferred that only a single towel be used to cover the leg support device 10 providing a more comfortable resting place for the patient's leg 50 while greatly reducing the usage of sterile towels and thus reducing the cost of the operation procedure. Finally, and perhaps what is more important, following a surgical procedure the leg support device 10 of the present invention may merely be inserted into an autoclave or the like for sterilization thereof so that it is ready for use with another patient without requiring a great expenditure of time and energy to prepare the device for reuse. The leg support device 10 of the present invention thus provides a substantial improvement over those methods and procedures for elevation of patients legs as found in the prior art.

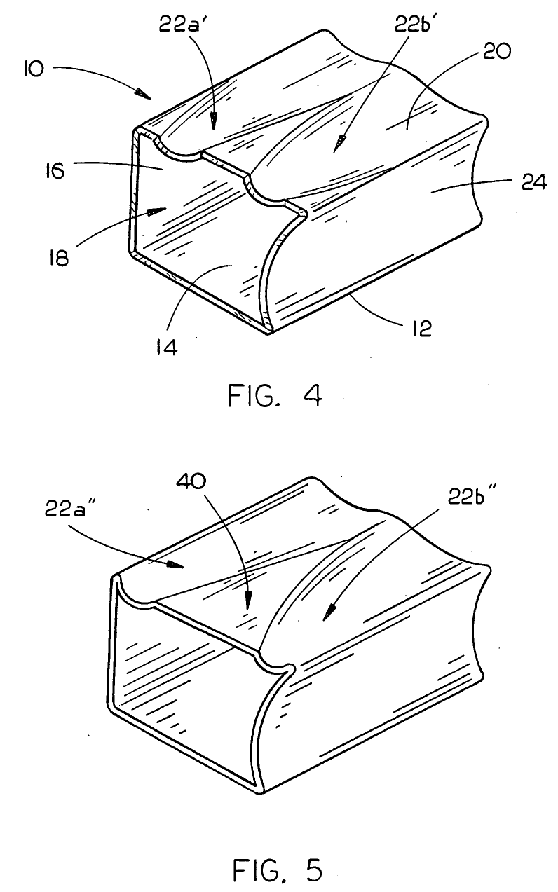

Two other embodiments of the present invention are shown in FIGS. 4 and 5 as including modifications to the first concave support wall 20. Specifically, as shown in FIG. 4, the concave sections 22a′ and 22b′ would include generally end-adjacent tapering sections which are formed in the first concave support wall 20. These tapering sections are formed by extending the generally flat portions of first concave support wall 20 outwards from the central area between concave sections 22a′ and 22b′ and from the outer edges of first concave support wall 20 while keeping the overall depth of the concave sections 22a′ and 22b′ approximately constant. This tapered designed embodiment is particularly adapted for supporting the lower leg portion of an individual using the leg support device 10 of the present invention, as the calf of the individual would fit within the broader portion of concave sections 22a′ and 22b′ adjacent one end of the first concave support wall 20 and the ankle section of the leg would comfortably fit within the tapered sections of concave sections 22a′ and 22b′ thus safely, securely and comfortably supporting the lower leg of the patient.

Likewise, as shown in FIG. 5, the center section 40 of first concave support wall 20 would be extended outwards from the middle of said first concave support wall 20 in a generally horizontal plane to provide separation of the two concave sections 22a″ and 22b″, each of which would maintain approximately the same depth of the concave section along the length thereof. In the case of an individual using a single leg support device 10 to support both legs, the embodiment shown in FIG. 5 is particularly designed to separate the legs from one another while still permitting the legs to be safely and securely supported on the leg support device 10. Of course, it should be noted that many different variations of curvatures and tapering section designs may be used with the present invention so long as the intended functional purpose of supporting an extremity is maintained and performed.

One concern found in connection with the present invention is that the edges of base opening 18 may be unintentionally sharp due to the metal surface being exposed, as shown best in FIGS. 1 and 3. The inventor's solution is to bevel or round the edges of the base opening 18 to eliminate all sharp edges, and the same elimination of sharp edges is intended to be performed on each of the edges of the rectangular box 12 in order to prevent accidental injury to the patient with whom the leg support device 10 of the present invention is being used. Many different types of protection may be used with the corners of the rectangular box 12 such as the fitting of pliable materials such as rubber or fabric over the edges, however, it has been found that a simple rounding of the edges is sufficient to remove the majority of the danger of accidental injury incurred by use of the present invention and this simple rounding of the edges does not detract from the feature of the present invention that it can be cleaned in an autoclave or the like. Other variations and modifications would be understood by those skilled in the art of metalworking.

It is to be understood that numerous modifications, additions and substitutions may be made to the leg support device 10 of the present invention which falls within the intended broad scope of the above description. For example, the size, shape, and construction materials of the present invention are not critical to the present invention so long as the functional characteristics of the invention are maintained. Additionally, the thickness of the walls of the rectangular box 12 of the present invention may be modified and/or changed depending on the size and weight of the patient's leg to be supported and, likewise, the size, depth, and extent of the concavities of the present invention may be modified or changed to accommodate variations in patients' leg size. Furthermore, although the present invention has been described as including three concave walls for supporting a leg thereon, the invention may be modified to include only two concave walls or even a single concave wall, depending on the needs of the end user of the present invention. Finally, although the present invention has been described for use in connection with patients' legs, it may also be used in connection with other extremities of patients, including arms, and may be used in veterinary situations for supporting of animal extremities, these applications to be understood by those skilled in the art of surgical and veterinary services.

There has thus been shown and described a leg support device for surgical procedures which accomplishes at least all of its intended objectives.

I claim:

1. An extremity support device comprising:
a box constructed of a rigid, non-permeable material, said box having at least six sides;
a first set of two sides of said at least six sides being positioned generally opposite and generally parallel with one another and having generally similar lengths;
a second set of two sides of said at least six sides being positioned generally opposite and generally parallel with one another and having generally similar lengths;
a first side of said first set having at least a portion thereof which is generally flat such that said box resting on said first side of said first set on a supporting surface is generally stable;
a second side of said first set including a generally concave wall surface extending substantially the length of said second side and having a generally cross-sectionally concave shape generally adapted to receive and releasably retain an extremity of a person therein;
a first side of said second set having at least a portion thereof which is generally flat such that said box resting on said first side of said second set on a supporting surface is generally stable;
a second side of said second set including a generally concave wall surface extending substantially the length of said second side and having a generally cross-sectionally concave shape generally adapted to receive and releasably retain an extremity of a person therein; and
said length of said sides of said first set being different than said length of said sides of said second set such that said second side of said first set is supported at one height above said first side of said first set when said box is resting on said first side of said first set and said second side of said second set is supported at another height above said first side of said second set when said box is resting on said first side of said second set.

2. The extremity support device of claim 1 wherein said box is generally hollow with an interior volume and at least one of said at least six sides being open to the interior volume of said box such that said box may be sterilized both externally and internally.

3. The extremity support device of claim 1 further comprising:
said box being generally hollow with an interior volume;
a third set of two sides of said at least six sides being positioned generally opposite and generally parallel with one another and having generally similar lengths;
a first side of said third set having at least a portion thereof operative to provide a resting surface such that said box resting on said first side of said third set on a supporting surface is generally stable and another portion thereof which is open to the interior volume of said box such that said box may be sterilized both externally and internally; and
a second side of said third set including a generally concave wall surface extending substantially the length of said second side and having a generally cross-sectionally concave shape generally adapted to receive and releasably retain an extremity of a person therein.

4. The extremity support device of claim 1 wherein said second side of at least one of said first and second set further comprises two generally longitudinal generally concave wall surfaces each extending substantially the length of said second side of at least one of said first and second set each having a generally cross-sectionally concave shape generally adapted to receive and releasably retain an extremity of a person therein.

5. The extremity support device of claim 1 wherein said box is constructed of metal.

6. The extremity support device of claim 1 wherein said box has a height dimension, a length dimension and a width dimension, said height, width, and length dimensions each being different from one another such that rotation of said box to rest said box on a different one of said first sides of said first and second sets of said at least six sides results in the topmost second side of said first and second sets being supported at a different height to facilitate adjustability of the height of said second side for the extremity to be supported.

7. An extremity support device comprising:
a generally rectangular box constructed of a generally rigid, non-permeable material and having six sides;
at least two extremity support sides of said six sides having generally longitudinally extended generally concave wall surfaces having a generally cross-sectionally concave shape generally adapted to receive and releasably retain an extremity of a person therein;
at least two box support sides of said six sides each opposite one of said at least two extremity support sides having at least a portion thereof which is generally flat such that said generally rectangular box resting on one of said at least two box support sides on a supporting surface is generally stable; and
said generally rectangular box having different height, width, and length dimensions such that resting said generally rectangular box on a first one of said at least two box support sides results in a first one of said at least two extremity support sides being supported at one height and rotation of said generally rectangular box to rest said generally rectangular box on a second one of said at least two box support sides results in a second one of said at least two extremity support sides being supported at a different height than the height of said first one of said at least two extremity support sides to facilitate adjustability of the height of at least two extremity support sides for the extremity to be supported.

8. The extremity support device of claim 7 wherein said generally rectangular box is generally hollow with an interior volume and at least one of said six sides being open to the interior volume of said box such that said box may be sterilized both externally and internally.

9. An extremity support device comprising:
a generally rectangular hollow box constructed of a generally rigid, non-permeable material and having an interior volume and six sides;
said six sides including three extremity support sides each having generally longitudinally extended generally concave wall surfaces having a generally cross-sectionally concave shape generally adapted to receive and releasably retain an extremity of a person therein;
said six sides further including three box support sides each opposite one of said three extremity support sides, said three box support sides each having a portion thereof operative to provide a resting surface such that said generally rectangular box resting on one of said three extremity support sides on a supporting surface is generally stable;
at least one of said three box support sides further including a portion thereof which is open to the interior volume of said box such that said box can be sterilized both externally and internally; and
said generally rectangular box having different height, width, and length dimensions such that resting said generally rectangular box on a first one of said at least two box support sides results in a first one of said at least two extremity support sides being supported at one height and rotation of said generally rectangular box to rest said generally rectangular box on a second one of said at least two box support sides results in a second one of said at least two extremity support sides being supported at a different height than the height of said first one of said at least two extremity support sides to facilitate adjustability of the height of at least two extremity support sides for the extremity to be supported.
